(19) United States
(12) Patent Application Publication
(10) Pub. No.: US 2012/0065691 A1
(43) Pub. Date: Mar. 15, 2012

(54) SPINAL IMPLANT ASSEMBLIES

(76) Inventor: Peter M. Simonson, Longboat Key, FL (US)

(21) Appl. No.: 13/197,728

(22) Filed: Aug. 3, 2011

Related U.S. Application Data
(60) Provisional application No. 61/370,427, filed on Aug. 3, 2010.

(51) Int. Cl. A61B 17/86

(52) U.S. Cl. 606/309

(57) ABSTRACT

A bone screw with self-tapping exit flutes as well as tools for pulling a spinal rod through spinal system connector assemblies. The self-tapping exit flutes are preferably a cutting flute at the threaded end of a bone screw closest to the bone screw shank. To facilitate insertion of spinal rods into connector assemblies, a flexible thread or wire is attached to the forward end of a spinal rod. This flexible thread is then attached to the rear end of a needle-like probe. The probe helps the surgeon thread the rod into and through connector apertures. To further assist this threading process, a pulling tool with a handle, shaft and a grabbing end can be used.

### CROSS-REFERENCE TO RELATED APPLICATIONS

This application claims the benefit of priority under 35 USC 119(e) of U.S. Provisional Application No. 61/370,427, filed on Aug. 3, 2010, which is incorporated herein by reference in its entirety for all purposes.

### FIELD OF INVENTION

The present inventions relate to devices used in orthopedic spinal surgical procedures. Specifically, the inventions improve upon spinal implant assemblies with a self-tapping exit flute for easier bone screw removal and a tool for pulling the spinal rod through spinal connector assemblies.

### BACKGROUND OF THE INVENTION

Back pain is a commonly reported medical aliment. It is most frequently associated with degenerative changes in the spinal vertebra. Most of the 30 million U.S. patients who report back pain each year resolve their pain with conservative treatment (e.g., rest and exercise). Nonetheless, approximately 15 percent or 4.5 million fail conservative therapy and are left with debilitating pain. Out of these, approximately 500,000 people opt for spinal surgery. In addition to alleviating pain, spinal surgery seeks to minimize damage to adjacent supportive muscle and skeletal components. Although other dynamic treatment options are becoming available, spinal fusion is the most common surgical procedure.

Several techniques and systems have been developed for correcting and stabilizing the spine (e.g., to facilitate spinal fusion). Over the years, spinal and orthopedic implants have evolved toward progressively stronger, stiffer and better devices, as it is presumed that increased construct rigidity optimizes bone fusion and provides more rapid and robust healing. The most widely used systems use a bendable rod that is placed longitudinally along the length of the spine. Such a rod is bent to follow the normal curvature of the spine whether it is the normal kyphotic curvature for the thoracic region or the lordotic curvature for the lumbar region. In such a procedure, a rod is attached to various vertebrae along the length of the spinal column by a number of bone anchor assemblies. A bone anchor element may be a hook that engages the vertebra laminae or a spinal bone screw threaded into the vertebral bone.

In traditional rigid stabilization systems, the rod is situated on opposite sides of the spine or spinous processes. Numerous bone screws are then screwed into the pedicles of the vertebral bodies. Rods are affixed to these bone screws so corrective and stabilizing forces are applied to the spine. After stabilization, the vertebra is typically decortified where the outer cortical bone is removed to provide a foundation for bone grafts. Over time, these bone grafts fuse the damaged vertebrae together.

A good example of rod spinal fixation is the TSRH® Spinal System sold by Medtronic Sofamor Danek Inc. The TSRH® Spinal System incorporates elongated rods, hooks, screws and bolts that are configured to create segmented constructs through the spine. In earlier versions, the vertebral hooks and bone screws were perpendicularly attached below the spinal rod at a fixed orientation. When introduced, the TSRH® Spinal System was a significant advance over prior systems. It provided the surgeon far more versatility, stronger fixation and easier implantation. Nonetheless, it also required the surgeon to make significant changes to the contour of the rod. If bent correctly, a bone fastener, such as a hook or screw, could be connected to the rod through lateral eyebolts adjacent to the rod.

One drawback to the original TSRH® spinal systems was that these lateral eyebolt fasteners met the rod at various angles making installation of the spinal rod very difficult. To solve this problem, the TSRH® Variable Angle Screw in U.S. Pat. No. 5,261,909 by Sutterline et al. utilized the same TSRH® eyebolt to connect the spinal rod but incorporated a washer. This washer engaged the spinal rod through a groove on one surface of the washer. More importantly, it added two opposing radially splined surfaces so that the fastener could move at variable angles relative to the spinal rod. Finally, a nut threaded onto the post of the eyebolt clamped all the components together to complete the assembly. Such a variable angle screw configuration allowed the bone screw to pivot in a single plane parallel to the plane of the spinal rod thereby eliminating one of the many angles that needed to be negotiated to meet the rod.

The variable angle screw system of U.S. Pat. No. 5,261,909 presented a significant advance over the prior rod-based implant system. It was also relatively compact and required a minimal number of parts, yet was able to accomplish a solid fixation of the bone fastener so that the rod had a wider range of angular orientations. A drawback, however, was that the eyebolt-nut combination required side tightening of the nut to clamp the systems together. This side tightening required a large surgical area around the spine to accommodate a wrench. To address this difficulty, a top-tightening assembly was disclosed in U.S. Pat. No. 5,282,801 to Sherman. In this patent, the clamp assembly replaced the eyebolt and nut with a T-bar against the head of the variable angle bone fastener. While the original TSRH® Spinal System relied upon tightening a nut against the variable angle bone screw, the top-tightening approach of U.S. Pat. No. 5,282,801 utilized a set screw that pushed the spinal rod into an interlocking washer, and ultimately against a complementary spline face of the variable angle screw. With this system, the variable angle capability was retained while a useful top-tightening feature was added.

With the addition of the top-tightening capability, the TSRH® Spinal System provided surgeons with a great deal of flexibility in the placement and orientation of bone fasteners. Although these variable angle components now greatly reduced the need to manipulate and bend the spinal rod, a certain amount of shaping or contouring of the spinal rod was still required. Specifically, the rod had to be shaped so that the point of attachment of the bone fastener to the rod was the same distance from the vertebral body as the splined or inter-digitating portion of the bone fastener. To do so, a vertical or height adjustment of the bone screw was necessary to ensure that the variable angle components were properly aligned with each other when the assembly is clamped together. If there was height difference between the bone screw and the rod, it was made up by threading or unthreading the bone screw either up or down.

To overcome this bone screw adjustment drawback, a system was developed to provide vertical adjustability. If the height difference between the rod and bone screw was close, the new system would allow a rod to be situated at any distance from the spine and/or oriented with a pre-set contour regardless of the location of the fastener. With the addition of a complementary splined surface to the bone washer described in U.S. Pat. No. 5,643,263 to Simonson, the TSRH® Spinal System now allowed a surgeon to easily engage a bent spinal rod to any type of fixation element for final tightening. Furthermore, various sized rods and screws could now be fixed in almost any angulation. With its vertical height adjustment ability, the widest angulations of any spinal bone anchor system can be met. Furthermore, it can also engage a rod or bone screw of any size or configuration without switching its connectors. The current TSRH® Spinal System allows a surgeon to easily engage a bent spinal rod to any type of fixation element.

FIG. 1 shows a spinal system assembly 2, more specifically a current design of the variable height TSRH® Spinal System. The spinal system assembly 2 comprises a rod 20, a bone screw 18 and a connector assembly 5, including a rod washer 4 with an oversized aperture 6 and bone washer 8 also with an oversized aperture 10. Like the earlier TSRH® Spinal System, the rod washer 4 has radially splined ridges 12 (FIG. 1C) facing the bone screw washer 8. As mentioned, the Simonson design added another complementary radially splined surface 12 to the bone screw washer 8. In combination, the spinal system assembly 2 moves at variable angles relative to the spinal rod with both the rod washer 4 and bone screw washer 8 able to rotate 180° with respect to one another. With an additional elongated smooth shank 14 and threads 16, the bone screw 18 becomes a bolt. The connections between the rod 20 and bone screw 18 are now a swivel that can be moved up and down 22 over the shank 14 and horizontally over the rod 20. With this invention, the rod and bone screw can be in differing angular positions. Furthermore, the one sized bone screw washer aperture 10 or bore can receive a bone screw shank 14 of any size and thicknesses with different sized interface washers. As such, any distance between the rod 20 and bone screw 18 can now be met. Adapting this type of bone fastener to the TSRH® Spinal System allows the bore diameters of both the rod 20 and bone screw 18 to be effectively reduced as the rod 20 and washers 4, 8 are pressed together by a top-tightening set screw 24. When the rod and bolt washers 4, 8 have been properly positioned over the rod 20 and bone screw 18, they are tightened by the set screw 24. When the set screw 24 forces the rod 20 toward the bone screw washer 8, the entire assembly becomes locked against any movement. Adjustments can then be made by loosening the set screw 24 and re-tightening it when the preferred position is reached. When properly adjusted, the set screw 24 is tighten and snapped off.

The rotation ability of the spinal system assembly 2 is also shown in FIG. 1. A rod 20 is positioned in the aperture 6 of the rod washer. The shank 14 of the bone screw 18 is positioned through the aperture 10 of the bone screw washer 8. Since the apertures 6, 10 of the rod washer 4 and the bone screw washer 8 are larger than the rod 20 and bone screw 18, they both can rotate 26, 28 relative to each other, respectively. With this flexibility between the rod 20 and a plurality of bone screws 18, the connector assembly 5 can be moved 30 along the rod 20. With such a configuration, the rod 20 and bone screw 18 can be in differing angular positions because the rod washer 4 and bone screw washer 8 can now rotate relative to each other. The linear distance between the rod and bone screw threads can also be adjusted because of the variability provided by the apertures. With this ability of the spinal system assembly to rotate 32 with respect to the rod and bone screw washers, the radial washers give the TSRH® Spinal System rotation in the sagittal plane 34.

Along with variable-sized washers 4, 8 and the 160° medial-lateral flexibility of the bone screws, the TSRH® Spinal System can also undergo a medial-lateral adjustment. In doing so, the engagement of laterally placed screws is now possible. This is especially important during multi-level rod constructs between the vertebrae. With the radially splined ridges 12, various sized washers and the smooth bone screw shank 14, the anatomic placement of pedicle screws can be made with minimal rod contouring. Such a configuration also minimizes any forced preloading or stressing of the screw-to-rod interface. With respect to other assemblies, no other spinal system assembly has all of these characteristics and abilities.

As the prior art illustrates, spinal fixation devices are constantly being improved. Whereas a rod 36 shown in FIG. 2 of a different assembly is easily top-loaded, the rod 20 of the TSRH® Spinal System is threaded through the aperture 6 of the rod washer 4. A need exists to assist the rod 20 of the TSRH® Spinal System to be easily threaded through the rod washer aperture 6.

Another common issue among all spinal system assemblies is bone screw pullout. After extensive wear, bone screws may weaken or begin to pullout. As a result, spinal system assemblies may begin to slip along their rods and bone screws. When the vertebral bone is strong and healthy, the initial fixation of traditional spinal and orthopedic screws is excellent, with more than adequate strength to resist pullout. With dense, sclerotic or osteoporotic bone, micro-motions resulting from the normal range of motion with the skeletal system may lead to a progressive degradation from the initially implanted state. Should the bone fail to heal, these micro-motions can persist and cause the metallic screw to oscillate within the softer cancellous bone. When subjected to persistent toggling, the modulus mismatch of the metal to cortico-cancellous bone interface may cause the bone screw to become loose.

A lot of time and preparation typically goes into inserting a bone screw into the cortical and cancellous bone of the vertebra. This includes preparing the pedicle canal, determining the screw length and tapping the pedicles with screws. This is particularly true for dense, sclerotic or osteoporotic bone. In healthy bone, some pedicle screws with self-tapping entry flutes at their distal or cutting end can easily penetrate the bone until its last thread is flush with the bony surface (see FIG. 2).

So as to not obscure screw alignment and placement, decortication is usually performed after the bone screws are set. When decortication is complete, cortico-cancellous bone grafts are firmly pressed in place to form a bone fusion bed. Although there are many types of segmental spinal fusion (dorsolateral intertranverse, posterior lumbar interbody, anterior lumbar interbody, etc.), the basic technique is very similar. Prior to placement of the bone graft, a fusion bed is thoroughly prepared by decortication of the bony surface to expose the underlying cancellous core. This usually includes the transverse processes, the facet joints and the par interarticularis. Decortication is performed with a variety of instruments such as a burr or curette. For a short-term segment fusion, unilateral posterior iliac crest harvesting provides adequate bone for grafting. For longer complex fusion, bilateral iliac crest bone harvesting may be necessary. Such bone graft is carefully applied to the decorticated surfaces in a continuous fashion, forming a bridge from one segment to the next.

A portion of the fusion process can be seen in FIG. 2. Once the bone screw 18 is properly screwed into place and before the assembly connector 5 and rod 20 are attached, decortication 38 is performed. With the reduced bone-screw interface of the TSRH® Spinal System, most of the bone facet areas around the smooth and raised bone screw shank 14 can be easily decortified. With a multi- or poly-axial assembly with a bulb type connector 40, decortification around the facets is a little more difficult because the bulb 40 and bone screw are loaded together as one piece. The bulb 40 obscures much of the facet 42. In contrast, a reduced profile of the shanks 14 of the spinal system assembly 2 allows the surgeon to adequately visualize and decorticate the bony elements in the lateral spinal gutter with screws 18 or shank 14 in place.

After decortication, cortico-cancellous bone graft 44 is firmly pressed on the bone fusion bed prior to the placement of the connectors 5 and rod 20 constructs. After the rod, connectors and bone screw are assembled; the normal curvature is obtained through segmental distraction and compression of the spine. After several adjustments and provisional tightenings, the surgeon performs the final tightening.

Over time, the cortico-cancellous bone graft fuses the vertebrae together. While the vertebrae are fusing, the main objective of spinal implants is to stabilize the spine until such time that fusion is complete. Once fused, the spinal implants become secondary. In general, most spinal implants are left behind and not removed because of the risk of surgery to do so. There are, however, many situations that require implant removal and this is particularly true during bone screw pullout. Classically, screw pullout, dislodgement and breakage are the main cause of implant failure. Such bone screw failure results in a weakening of mechanical strength for the overall construct. It also lowers the biological potential for bone healing. A need therefore exists to more easily remove bone screws especially through the newly laid cortico-cancellous fusion bed.

In summary, there is a need in the industry for continual improvements in devices, implants and tools used in orthopedic spinal surgical procedures. In particular, improvements need to be made in removing existing spinal system assemblies, especially bone screws. For spinal system assemblies, where the rod is threaded through connector apertures, a need exists to help pull the rod through the connectors. The present invention describes improvements in these areas for many makes and models of spinal implants on the market today.

### BRIEF SUMMARY OF THE INVENTIONS

The present invention provides improvements to bone screws, rods and tools used in orthopedic spinal surgical procedures. Specifically, the present invention improves the spinal system assemblies with one or more exit flutes on the bone screw and tools for pulling the spinal rod through the spinal system assembly.

A self-tapping exit flute is a cutting flute at the proximal end of a bone screw (i.e., next to the bone screw shank). If bone screw removal is needed, the self-tapping exit flute of the present invention permits a cleaner removal of the screw through the previously tapped cortical and cancellous bone of the vertebra and, more importantly, through the newly laid and fused bone created by the cortico-cancellous bone graft. Such a self-tapping exit flute may prevent cracking or disruption of the bone fusion bed, especially if a healthy fusion bed is maintaining its strength and integrity.

A second embodiment pertains to spinal system assemblies where the rod is threaded through apertures of connectors or washers. Rather than pushing rods through the apertures, a pulling tool is used to help pull the rod through the connector washer apertures. One preferred embodiment is a flexible thread, string or wire attached to the distal end of a spinal rod. This flexible thread is attached to a needle-like probe. The probe helps the surgeon insert the flexible thread into and through the connector washer apertures. Once the probe is through the connector, an attachment mechanism on the probe connects to a pulling tool with a handle. In one embodiment, the distal end of the pulling tool attaches or snaps onto the probe by a hooking device. The surgeon then pulls the rod through the apertures of the connector washers by a rod puller. In combination with a pushing tool, a pulling tool may facilitate the placement of the spinal rods through the connector washers.

### BRIEF DESCRIPTION OF THE DRAWINGS

FIG. 1 shows a perspective view of the prior art TSRH® Spinal System described in U.S. Pat. No. 5,643,263 to Simonson with FIGS. 1A, 1B and 1C showing more detail of the rod and bone screw washers.

FIG. 2 shows a prior art bulb type bone anchor assembly and a variable height TSRH® spinal system assembly in use during spinal fusion surgery.

FIG. 3A shows a variable height bone screw of the present invention with self-tapping entry and exit cutting flutes at its proximal and distal ends.

FIG. 3B shows a close up view of the self-tapping exit cutting flutes of the present invention on the proximal end of the bone screw.

FIG. 3C shows the self-tapping exit flute cutting through pedicles of a fusion bed upon removal from the vertebral body.

FIG. 4A shows a perspective view of an aperture probe of the present invention being threaded an aperture.

FIG. 4B show an aperture probe and its thread attached directly to the spinal rod.

FIG. 4C shows an aperture probe using a thread looped through the spinal rod with a ball and hook connection on its forward end.

FIG. 5A shows a rod puller tool with a handle.

FIG. 5B shows a hook embodiment of the rod puller tool.

FIG. 5C shows a slot embodiment of the rod puller tool.

FIG. 6 shows a schematic of the rod puller attached to an aperture probe pulling a spinal rod through a spinal system assembly.

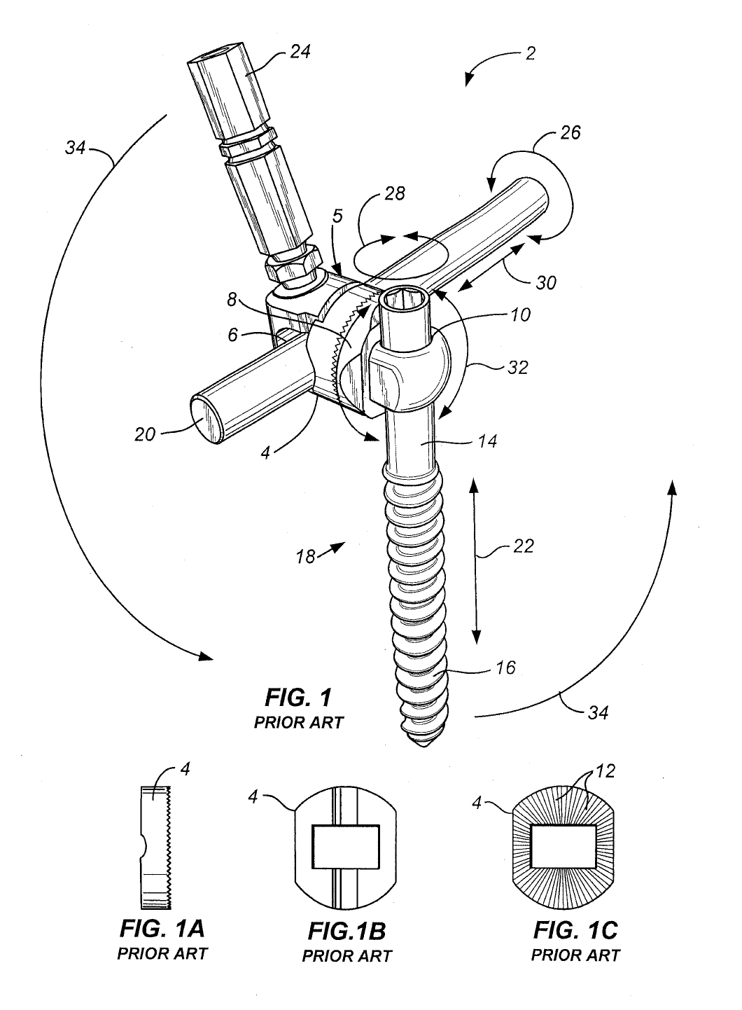

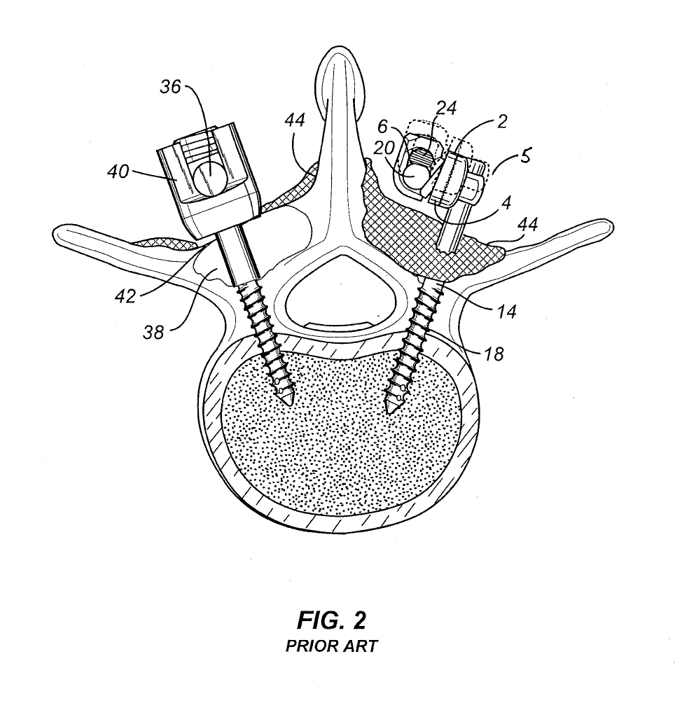

### DETAILED DESCRIPTION OF THE INVENTION

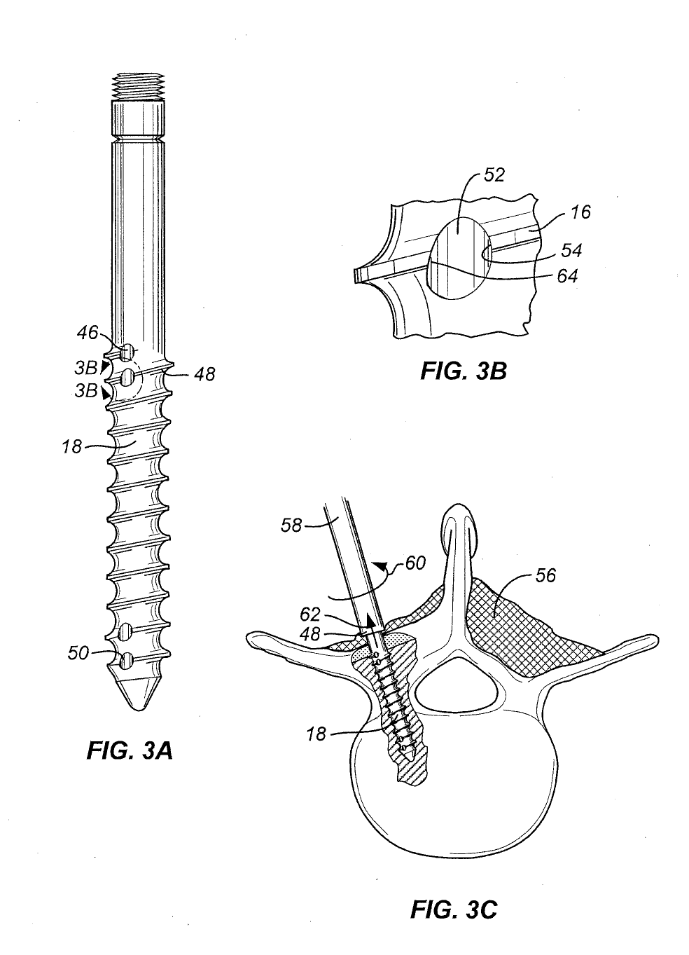

A self-tapping exit flute of the present invention is shown in FIG. 3A. In FIG. 3A, a self-tapping exit flute is a flute 46 cut into the proximal end thread 48 of a bone screw 18. Preferably, there are one or more flutes 46 positioned around the proximal end threads 48 of a bone screw 18. In one embodiment, such exit flutes 46 are preferably on bone screws that also have entry flutes 50. When such a bone screw possesses both entry 50 and exit 46 flutes, it can be truly self-tapping in either direction. Self-tapping is the ability of a screw to advance when turned and creates its own thread. It is created when a gap is cut into a thread to disrupt the continuity of the thread on the screw. The gap is usually the depth or height of the thread.

A close-up of the self-tapping exit flute is shown in FIG. 3B. A gap 52 is cut into a thread 16. When the bone screw threads 16 are entering the bone in a clock-wise direction, the entry cutting edge 54 of both the entry 50 or exit 46 flutes cuts their own thread as the screw is driven into the bone. In brief, a self-tapping screw acts as its own drill to clear away material but it also forms reverse threads in the bone. While entry flute 50 performs most of the cutting during entry, the cutting edge 54 of the exit flute may help the insertion of the bone screw 18 by removing the remaining debris or extruded fragments caused by either earlier drilling, tapping or screwing.

Referring to FIG. 3C, a meticulously prepared fusion bed or newly formed cortical bone 56 as shown in FIG. 3C will enhance the likelihood of a bone fusion. Once laid, a fusion bed starts the process of fusing a select number of vertebrae over time.

As illustrated in FIG. 3C, the removal of the bone screw 18 by a drill or driver 58 turns 60 the bone screw 18 upward 62 through the fusion bed or newly formed cortical bone 56. As shown in FIG. 3B, the exit flute 46 cutting edge 64 slices and cuts cleanly through the fusion bed or, if given enough healing time, the new cortical bone 56. Otherwise, a bone screw without an exit flute 46 may fracture either the fusion bed or new cortical bone 56. The exit flute 46 of the present invention makes the bone screw 18 self-tapping in the reverse direction 60. With such an invention, the fusion bed or cortical bone 56 remains intact and can continue to fuse or support the existing fused vertebrae.

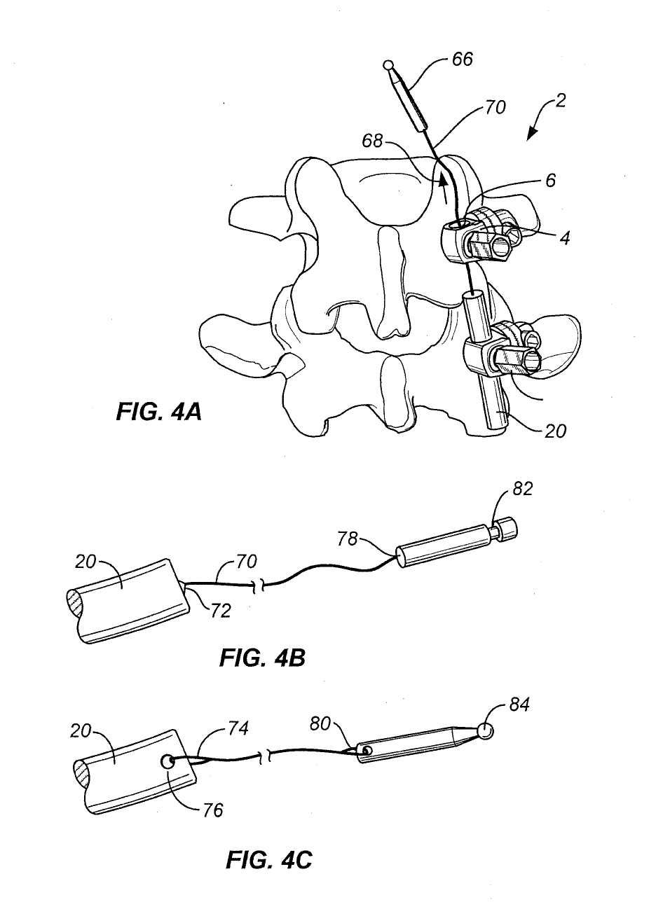

FIG. 4A-C shows embodiments of the rod puller invention. FIG. 4A shows how a probe of the present invention is pulling 68 a spinal rod 20 through its spinal system assemblies 2. In one embodiment, the rod puller consists of a probe 66 that is threaded through the aperture 6 of the rod washer 4. The probe 66 is attached to a flexible thread 70 that is in turn attached to the spinal rod 20. In one embodiment, the probe 66 may preferably be a suture needle. The flexible thread 70 can be preferably made from absorbable or non-absorbable suture material selected from a group including polyglycolic acid, polylactic acid, and polydiosanone. Absorbable materials are preferable since such flexible thread 70 may be cut to remove the probe 66, leaving perhaps a tiny amount of thread attached to the rod 20. The flexible thread 70 may also be selected from a group of non-absorbables such as nylon and polypropylene. The flexible thread 70 may be coated with antimicrobial substances to reduce the chances of wound infection. Good suture type materials include MONOCYRL™ (poligelcaprone), VICRYL™ (polyglactin), PDS™ (polydiodioxanone) made by ETHICON (Johnson & Johnson). For greater pulling strength, the flexible thread 70 may be made of stronger fibers such as aramid fabrics like DuPont's Kevlar® or Nomex® polyethylene fibers. For superior pulling strength, the flexible thread 70 can also be wire made from metals such as stainless steel or titanium. The primary purpose of such a probe 66 is to help the surgeon thread the rod 20 through the aperture 6 of the rod washer 4. Once through the aperture 6 of a rod washer 4, the surgeon can pull the probe 66 by applying pulling forces 68 to the rod 20. Alone or in combination with a pushing tool, the rod 20 may be more easily threaded through multiple spinal system assemblies 2 with the present invention.

There are various ways to connect the flexible thread 70 to the spinal rod 20. As shown in FIG. 4B, it may be directly attached to the forward end of the spinal rod 20 with either adhesive material 72 such as glue or epoxy-like materials. The thread 70 may also be looped and tied 74 through a hole 76 at the forward end of the spinal rod 20 as shown in FIG. 4C. It can also be attached to the rear end 78, 80 of the probe 66 in a similar manner. There are also various embodiments of the forward end of the probe 66 that attaches to a pulling tool with a handle. In one preferred embodiment shown in FIG. 4B, the probe 66 may have a slot 82 that connects into a corresponding c-shaped puller. In another embodiment shown in FIG. 4C, the probe 66 may have a ball 84 at its forward end which connects to a hook-like device.

Although the rod 20 can be pulled through the apertures 6 of the connector assemblies 5 by a surgeon pulling on the probe 66, there may be circumstances to have additional pulling and gripping force. As shown in FIG. 4A, this is especially true when the rod 20 is threaded through multiple connector assemblies. In such cases, resistance of the rod 20 through numerous apertures 6 becomes so much greater that a pulling tool may help.

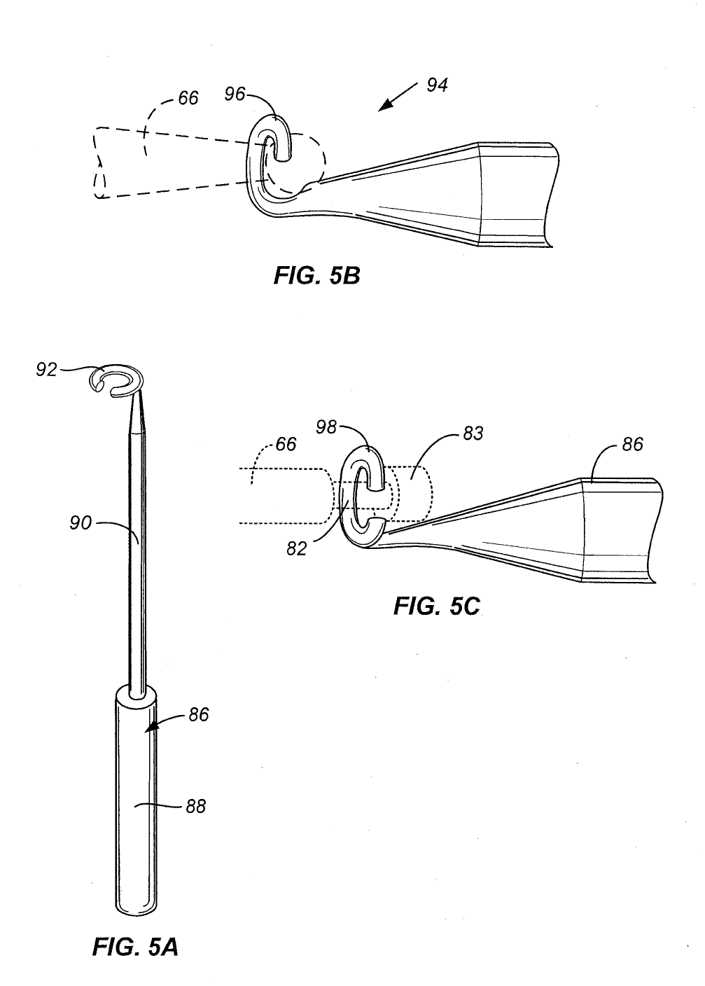

As shown in FIG. 5A, a rod pulling tool 86 preferably consists of a rubberized handle 88 for greater grip, a metal or plastic shaft 90 for greater leverage and a grabbing end 92. FIGS. 5B and 5C show various preferred embodiments of the rod pulling tool 86 especially its grabbing ends. In one preferred embodiment, FIG. 5B shows a hook and ball 94 configuration. A hook 96 built into the end of the rod-pulling tool 86 catches the ball end of probe 66 in FIG. 5B. When hooked, the surgeon simply pulls on the rod-pulling tool 86 to achieve enough leverage to help pull the spinal rod through either single or connector assemblies 5. Another grabbing end configuration is shown in FIG. 5C. FIG. 5C shows a slot 82 and head 83 on probe 66 snapping into a c-hook 98. Again, the c-hook 98 is attached to the rod puller tool 86 to assist the surgeon in pulling the spinal rod 20.

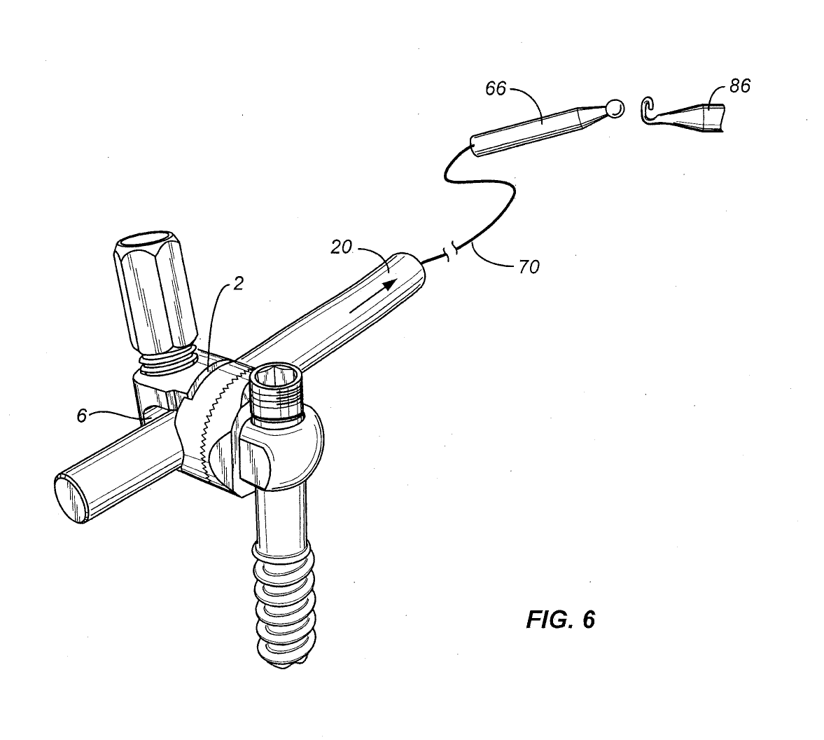

The entire rod puller assembly is shown in FIG. 6 with the spinal rod 20 being pulled by the rod puller 86 through which its probe 66 and its flexible thread 70 transfer a pulling force to the spinal rod 20 that facilitates its movement through the aperture of a connector assembly 5. When the spinal rod 20 is through the apertures 6 of all of its connector assemblies 5, the probe 66 along with its flexible thread 70 can be easily removed by cutting the flexible thread 70 near or at the forward end of the rod.

In the foregoing specification, the invention has been described with reference to specific preferred embodiments and methods. It will, however, be evident to those of skill in the art that various modifications and changes may be made without departing from the broader spirit and scope of the invention. Accordingly, the specification and drawings are to be regarded in an illustrative, rather than restrictive, sense; the invention being limited only by the appended claims.

What is claimed is:

1. A bone screw comprising a shank portion and a threaded portion, wherein one or more flutes are formed in said threaded portion in close proximity to said shank portion.

2. The bone screw of claim 1 wherein said flutes are made on the thread closest to said shank.

3. The bone screw of claim 1 further comprising one or more flutes on a portion of the threads furthest away from the shank.

4. A method for removing bone screws from cortico-cancellous bone comprising the steps of:
selecting a bone screw with a shank portion and threaded portion, wherein one or more flutes are formed on said threaded portion in close proximity to said shank portion;
attaching a driver to said bone screw with flutes;
turning said bone screw in a reverse direction with said driver;
cutting through cortico-cancellous bone with said bone screw in the course of removing said bone screw from said cortico-cancellous bone.

5. A medical device for pulling a spinal rod through apertures of spinal system assemblies comprising a flexible thread connected to a probe.

6. The medical device of claim 5 wherein said flexible thread is further attached to a spinal rod.

7. The medical device of claim 5 wherein said flexible thread is metallic.

8. A method for pulling a spinal rod through apertures of a spinal system assembly using flexible thread attached to both a spinal rod and a probe comprising the steps of:
inserting said probe through apertures of said spinal system assemblies;
pulling on said probe to thereby pull said spinal rods through said apertures.

9. A medical tool for pulling a spinal rod through apertures of spinal system assemblies comprising a shaft and a grabbing end.

10. The medical tool of claim 9 further comprising a handle attached to said shaft.

11. The medical tool of claim 9 wherein said grabbing end is a hook, snap or connector mechanism.

12. A method for pulling a spinal rod through apertures of spinal system assemblies using a pulling tool comprising the steps of:
hooking, snapping or connecting said pulling tool onto a probe attached to a spinal rod;
pulling on said attached probe using said pulling tool;
inserting said spinal rod through said apertures as a result of pulling said probe.
